(19) United States
(12) Patent Application Publication
(10) Pub. No.: US 2004/0171992 A1
(43) Pub. Date: Sep. 2, 2004

(54) Cream dispenser providing sequential dispensing means

(75) Inventor: Bent Formby, Santa Barbara, CA (US)

Correspondence Address: Waikiwi Corporation, 244 Oldwoods Road., Franklin Lakes, NJ, 07417, US

(73) Assignee: Waikiwi Corporation

(21) Appl. No.: 10/787,241

(22) Filed: Feb. 27, 2004

Related U.S. Application Data
(60) Provisional application No. 60/450,437, filed on Feb. 28, 2003.

(51) Int. Cl. A61M 5/00

(52) U.S. Cl. 604/244

(57) ABSTRACT

The specification relates to an apparatus for storage and dispensing of a topically applied medicament to an animal by a dosing regimen that helps to facilitate administration of uneven doses of a biologically active substance. The apparatus includes a blister pack 1, 4, 9 with a plurality of predetermined medicament-containing wells 2, 5, 10 where the wells 2, 5, 10 contain medicaments 3, 6, 11 incorporated within a solution suitable for topical administration and, wherein the medicament 3, 6, 11 contains at least one biologically-active compound. Indicia 13 are described to help facilitate administration including use of color indicia.

### RELATED APPLICATIONS

This application is a complete US application priority from U.S. provisional application No. 60/450,437 filed on Feb. 28, 2003.

### BACKGROUND

The present invention relates to a dispenser apparatus to store and dispense an aqueous solution containing a medicament which includes at least one biologically-active substance. More specifically, the invention relates to a blister pack which is used to store and dispense a cream for use in transdermal treatment of an animal.

Physiological requirements vary from individual to individual and even within an individual during the course of a lifetime. Further, various conditions may effect physiological requirements. For example, pregnant, lactating and menopausal women may have enhanced needs for certain nutrients, therapeutic agents or treatments and reduced needs, or even intolerance, for other nutrients, therapeutic agents or treatments.

Meeting the specific physiological requirements of humans and other animals may require the use of a detailed, often daily, regimen requiring administration of various biologically-active substances simultaneously or at differing times during a treatment period. Further, the type of medication, volume of active agent required and concentration of dose may also need to be altered during treatment.

Many attempts have been made to address this problem and address the needs of the patient by providing apparatus that allows the patient an unambiguous indication as to whether or not they are taking the appropriate dose/medication and frequency. Further attempts have also been made in this regard to address the requirements of storing biologically active substances in a stable manner, and allowing the dispensing party e.g. the pharmacist, to dispense an amount of medication that is tailored to the patients needs.

WO 01/07012 (Drugtech Corporation) discusses in depth existing methods of delivering biologically active agents to a patient. The publication describes many attempts to deliver such agents to a patient in a manner that addresses known problems. The publication further adds to the prior art by describing an apparatus that, in its simplest form, includes two recessed portions, each containing different dosage forms and written indicia to direct the patient as to when the dosage form should be taken, for example ‘AM’ and ‘PM’. Further embodiments describe this double recessed apparatus in the form of a strip or blister pack that contains multiple dosages in multiple recesses, all with written indicia stating when they should be used e.g. by days of the week or time of day. The publication and examples within describe treatments incorporating different tablets that contain the active agent. Other types of dosage form besides tablets are mentioned in passing however, no enabling disclosure is indicated for liquid or semi liquid dosage forms.

Great Britain Patent No. 2 228 922 (Alan John Gordon et al) describes another type of packaging device used for dispensing drugs of varying types and levels. The dosage form is a tablet and the packaging used is aimed at allowing a dispensing pharmacist the ability to tailor the treatment regime to the patient by inserting the desired tablets into specifically placed recessed portions on the packaging and then sealing the packaging by combining the two leaves of the packaging. The end result is a packet that contains tablets in a tailored arrangement that, via written indicia, gives the patient an indication as to which tablets to take when. No disclosure is made as to how creams may be used in the packaging.

A further example where packaging is used to give an indicator as to when a dose needs to be taken includes WO 98/22072 (The Proctor & Gamble Company). In the case of this publication, a complete package is described for orally taken medications including a blister card with visual indicia as to when each tablet should be taken, a patient information booklet, a calendar and other reminder aids.

The above prior art is directed to the delivery of drugs via the oral route in tablet form.

Publication WO 01/07012 (Drugtech Corporation) suggests that other dosage types may be possible, however, no examples or enabling disclosure is made as to how a cream might be used in such treatment regimes.

The administration route is important to drug efficacy. All drugs must be administered in such a manner that they reach the intended site in the body in an optimal concentration to achieve the desired effect at the proper time. Typically, drugs are taken orally or by injection to achieve the desired effect. However, the dose required for some treatments via the oral route can be significantly larger than is actually required as active agent is lost during normal digestion processes before it reaches the site.

One method that has been considered to address delivery issues is that of topically applied medications such as patches and creams. Despite much research, transdermal application is still not a mainstream method of delivering an agent in the same way that orally taken tablets are used.

Topical transdermal formulations for delivery of hormonal compounds are known and discussed in the patent literature, see for example the background discussion of U.S. Pat. No. 5,968,919 (Samour et al). Attempts have been made to address problems in transdermal delivery through use of skin penetration enhancing compounds such as that disclosed in U.S. Pat. No. 5,968,919.

Further problems associated with topical preparations besides delivery issues are that of practical issues such as packaging the cream in a manner that keeps the cream stable but also in a form that allows a measured dose of active agent. Packaging such as a tube of cream does not keep the cream stable if for example the lid is not closed properly. Further, the dose to be applied is not in a pre-measured form, hence variations may occur in the amount applied by the patient. Further, the amount of active agent in a cream tube (and the active agent itself) cannot be varied in a tube. A yet further problem with creams, particularly those presented in jars, is that they are easily contaminated, for example by material on the patient's fingers.

It is therefore an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, or in any country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### SUMMARY

As used herein, ‘animal’ refers to a human, mammal or any other animal.

‘Biologically-active substance’ refers to any substance or substances including a drug, active therapeutic substance, metabolite, medicament, hormone, steroid, vitamin, fatty acid, amino acid, sugar, carbohydrate, polypeptide or mineral, any substance used for treatment, prevention, diagnosis, cure or mitigation of disease or illness, any substance which affects anatomical structure or physiological function, or any substance which alters the impact of external influences on an animal, or metabolite thereof, and as used herein, encompasses the terms ‘active substance’, ‘therapeutic substance’, ‘agent’, ‘active agent’, ‘active therapeutic agent’, ‘drug’, ‘medication’, ‘medicine’, ‘medicament’ and the like, without limitation.

‘Dosing regime’ refers to systematic administration of multiple dosage units of a medicament or medicaments at designated times during a period of time including, without limitation, administration of multiple dosage units which are potentially confusing or impractical to administer to a patient.

‘Uneven dosing’ or ‘unevenly dosed’ refers to doses of a biologically active substance wherein at least one dose following the initial dose contains a different amount or type of biologically-active substance than the previous dose.

‘Dose’ refers to each individual release of substance into body tissue and varies in terms of concentration, volume and biologically-active substance.

‘Shelf stability’ or ‘storage stability’ refers to the ability of a substance to resist degradation or alteration in chemical, physical or biological properties while awaiting use during a period of at least six months.

‘Blister pack’ refers to a sheet with one or more wells, inside which is contained a medicament, which is sealed within the well by a second sheet. The construction of the blister pack is such that the sealing sheet is independently accessible, removable or breakable.

The term ‘well’ refers to any shape of recessed portion capable of containing a medicament within it and encompasses the terms ‘recessed portion’, ‘chamber’, ‘enclosure’ and the like, without limitation.

According to one aspect of the present invention there is provided a disposable dispensing apparatus for storage and dispensing of a topically applied medicament to an animal by a dosing regimen to facilitate administration of uneven doses of a biologically active substance, said apparatus including;

a blister pack having a plurality of predetermined medicament-containing wells;

characterised in that the plurality of wells contain medicaments and are grouped, the wells being arranged and grouped for use of the medicament in each well; and,

further characterised in that medicament is incorporated within a solution suitable for topical administration; and,

wherein the medicament contains at least one biologically-active substance.

According to a further aspect of the present invention there is provided a disposable dispensing apparatus for storage and dispensing of a topically applied medicament to an animal by a dosing regimen to facilitate administration of uneven doses of a biologically active substance, said apparatus including;

a blister pack having a plurality of predetermined medicament-containing wells;

characterised in that the plurality of wells contain medicaments and are grouped, the wells being arranged and grouped for use of the medicament in each well; and,

further characterised in that medicament is incorporated within a solution suitable for topical administration; and,

wherein the medicament contains at least one biologically-active substance; and,

further characterised in that the blister pack includes color indicia to further facilitate administration.

According to a further aspect of the present invention there is provided a disposable dispensing apparatus for storage and dispensing of a topically applied medicament to an animal by a dosing regimen to facilitate administration of uneven doses of a biologically active substance, said apparatus including;

a blister pack having a plurality of predetermined medicament-containing wells;

characterised in that the plurality of wells contain medicaments and are grouped, the wells being arranged and grouped for use of the medicament in each well; and,

further characterised in that medicament is incorporated within a solution suitable for topical administration; and,

wherein the medicament contains at least one biologically-active substance; and,

further characterised in that the solution provides color indicia to further facilitate administration and wherein at least the wells of the blister packet are substantially clear.

Preferably, the solution is a cream, gel, suspension, or semi-solid solution. It will be appreciated that creams may be made using a cream base, for example DMS base cream and oils such as castor oil. Biologically-active substances may then be added to the cream as required to form the medicament.

For the purposes of this description, reference will now be made to the term ‘cream’. This should not be seen as limiting.

Preferably, the apparatus includes a group of wells arranged in a plurality of rows of cream containing wells. Wells are preferably designed to be arranged in discrete and separate locations thus keeping groups of medicament identifiable visually or by feel from other groups on the blister pack or other blister packs.

Preferably, the wells are grouped for sequential use. By way of example, doses are arranged in a straight line, arc or any other shaped line.

Preferably, the blister pack includes indicia on the blister packet to indicate when the dose is to be taken.

In preferred embodiments, at least the top of at least one well recess is a transparent material and the cream color is used as indicia. Preferably the cream color is used to indicate when to take the dose although it will be appreciated by those skilled in the art that other indicia may also apply such as different dosages or biologically-active substances.

Preferably, the range of colors used either on the blister packet, in the cream or both form a recognisable sequence.

Preferably, the dose to be taken is varied depending on the biologically-active substance to be taken, the concentration and/or volume of biologically-active substance, and/or the time of day, week, month, quarter.

Preferably, time indicia includes markings selected from the group consisting of: AM, PM, morning, daytime, night-time, and combinations thereof.

Preferably, day indicia includes markings selected from the group consisting of: Monday, Tuesday, Wednesday, Thursday, Friday, Saturday, Sunday, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, one, two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one and combinations thereof.

Preferably, week indicia include markings selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52.

Preferably, month indicia include markings selected from the group consisting of: J, F, M, A, M, J, J, A, S, O, N, D, Jan, Feb, Mar, Apr, May, Jun, Jul, Aug, Sep, Oct, Nov Dec, January, February, March, April, May, June, July, August, September, October, November, December, and combinations thereof.

Preferably, quarter indicia include markings selected from the group consisting of: 1,2,3,4, JFM, AMJ, JAS, OND, and combinations thereof.

Preferably, each well has a seal that is independently accessible, removable or breakable. Most preferably, the seal securely retains the medicament in a stable and sterile form until ready for use.

Preferably, groups of wells include surrounding perforations. Perforations allow groups of well-containing medicaments to be separated from the whole blister pack.

Preferably, the blister pack also includes non-cream medicaments. For example, this may be done to administer a regime including both transdermal administration and oral administration i.e. a cream and a tablet.

Preferably, the apparatus further includes a container for receiving and storing a blister pack or packs.

Preferably, the container is divided into a plurality of discrete compartments and wherein at least one compartment is for receiving and storing a blister pack and at least one compartment is for receiving and storing further blister packs.

In one alternative embodiment, the blister pack is configured in a rolled formation such that as a strip of wells is pulled, the dose to be taken next is revealed from the container.

Preferably, the animal is a human however this should not be seen as limiting as it is envisaged that that people administering a treatment regime to an animal, for example a pet, may also benefit from the apparatus of the present invention. For example, a course of anthelmintic treatments for treatment of nematode worms in an animal which is applied by the owner.

In preferred embodiments, the medicament within each well contains an uneven dose and in one embodiment envisaged, each well contains a medicament that includes four or more different varieties of concentration, type or volume of biologically-active substance for each dose.

Preferably, the dose applied/used taken is one well per day.

Preferably, the blister pack contains doses of medicament in each well tailored to the dosage required by the patient. For example, hormone quantities in each dose are matched to those found in a women's typical 28 to 30 day menstrual cycle.

In one embodiment, the total number of doses included on a blister packet is for a twenty-eight day time period.

In an alternative embodiment, the total number of doses included on a blister packet is for a thirty day time period.

In one application envisaged by the applicant, the biologically-active substances are hormones and the quantity (concentration and/or volume) of medicament provided in each well is sufficient for the daily needs of a woman's natural monthly cycle.

It is envisaged by the applicant that the apparatus will be useful for a wide variety of applications including hormone treatments such as hormone replacement therapy.

It should be appreciated by those skilled in the art that there is provided an apparatus that provides a precise transdermal dose for the patient and practitioner and which can be easily administered by the patient. Further, the patient can quickly determine how many applications/dosages have been used, how many are left. Further, the dose regime can be tailored to the desired application with the dose concentration and volume able to be altered in each well.

### BRIEF DESCRIPTION OF THE DRAWINGS

Further aspects of the present invention will become apparent from the ensuing description which is given by way of example only and with reference to the accompanying drawings in which:

FIG. 1 is a plan view of one embodiment of the present invention showing a once-per-day dispenser for the administration of hormone containing cream where two rows of cream containing wells are used;

FIG. 2 is a side cross-sectional view taken along line 1 of FIG. 1 illustrating the cream containing wells shown in FIG. 1;

FIG. 3 is a plan view of a second embodiment of the present invention showing a twice-daily dispenser for the administration of hormone containing cream; and,

FIG. 4 is a plan view of a third embodiment of the present invention showing a once-per-day dispenser for the administration of hormone-containing cream.

### DETAILED DESCRIPTION

The apparatus of the present invention is a storage stable disposable dispensing apparatus which provides tailored delivery of a medicament to an animal and increases the ease with which a regime of the medicament is taken.

The present apparatus incorporates various types of blister packs. The blister packs are characterised by a plurality of single compartments referred to herein as ‘wells’. Each well contains a dose of medicament incorporated into an aqueous solution for use in transdermal applications. In key embodiments of the present invention, the aqueous solution is a cream in which the medicament is incorporated. The medicament may be an uneven dose and include one or more biologically-active substances and/or different concentrations of biologically-active substances and/or different volumes of biologically-active substances.

Each well retains a discrete dose of medicament which is stable inside the well until removed. In this manner, biologically active substances that may breakdown in air or in contact with other active agents, remain stable until ready for use. It is envisaged by the applicant that the time of stability may be comparable with oral medications such as tablets.

A further advantage of keeping the cream/medicament mix in a discrete well is that the mixture does not react with other active substances and thus form unwanted by-products or breakdown the desired active agent.

This arrangement, especially when used with indicia, eases administration of the medicament and allows for an exact dose to be taken rather than the guess work in the dose to be taken when obtaining cream from a jar or tube.

For the purposes of this description, reference will be made to the use of hormones as the biologically active substance contained within the wells. It should be appreciated by those skilled in the art that many other biologically-active substances can be incorporated within the same apparatus.

Further, the examples given are described with reference to a menstrual cycle application. It should be appreciated by those skilled in the art that many other applications are also possible and the examples described should not be seen as limiting.

In one example of the present invention, the apparatus has been found to be particularly advantageous for hormone treatments. In one embodiment the dose contained within wells is varied each week to match the natural hormone levels during a 28-day or 30-day menstrual cycle. In one embodiment the dose within the well is made up as shown in Table 1 below:

TABLE 1
Example Hormone Dose Regime

| | Week 1 | Week 2 | Week 3 | Week 4 |
|---|---|---|---|---|
| Ingredient | (wt %) | (wt %) | (wt %) | (wt %) |
| DMS Base Cream | 87.49 | 84.97 | 79.98 | 79.99 |
| Caster Oil | 10.00 | 10.00 | 10.00 | 10.00 |
| Micronised | 2.50 | 5.00 | 10.00 | 10.00 |
| Progesterone | | | | |
| Micronised 17-Beta | 0.01 | 0.03 | 0.02 | 0.01 |
| Estradiol | | | | |
| Total | 100.00 | 100.00 | 100.00 | 100.00 |

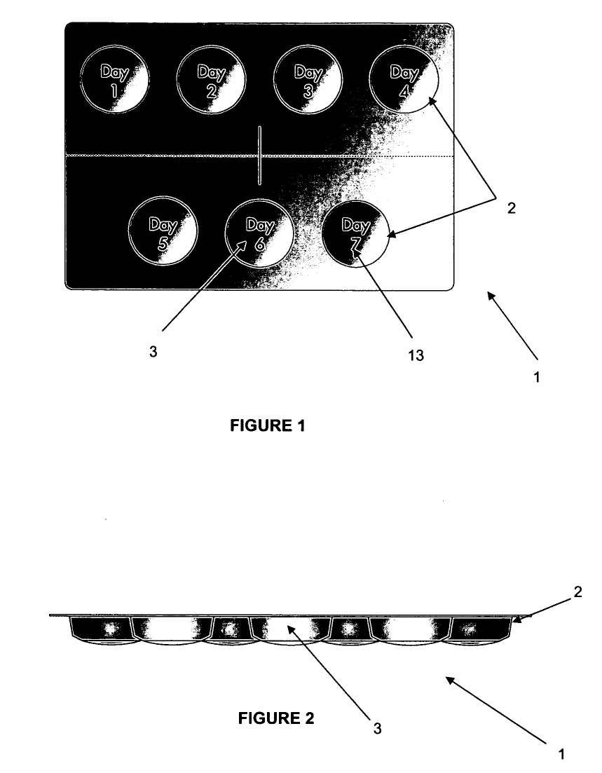

Referring to FIGS. 1 and 2, a first embodiment of a once-per-day dispenser for the administration of hormone containing cream is shown. The blister pack, generally indicated by arrow 1 contains two rows of wells 2. Each well 2 contains a specific concentration and ratio of hormones (‘the dose’ 3) to closely match that as found in a women's typical menstrual cycle. In this embodiment, the blister pack 1 contains seven wells 2 containing seven doses 3 corresponding to the number of days in a week. For a complete 28-day course of doses 3, four separate blister packs 1 are used. It is envisaged that the four blister packs will be incorporated into a container (not shown). This container may have further indicia to indicate to the user as to when to use each blister pack 1. In a further embodiment, the blister pack 1 may be in the shape of a roll with doses 3 in sequential order.

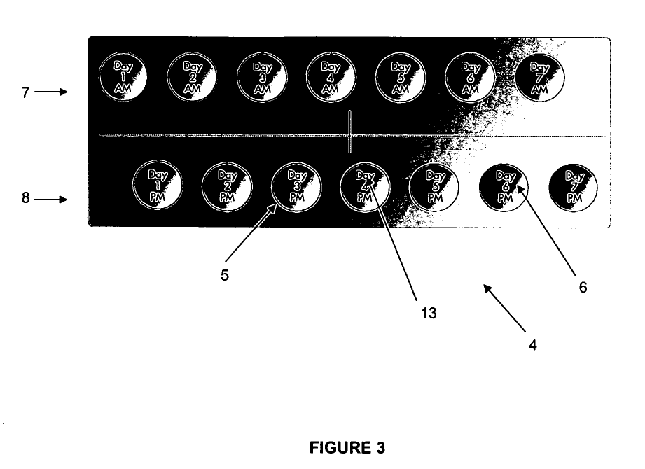

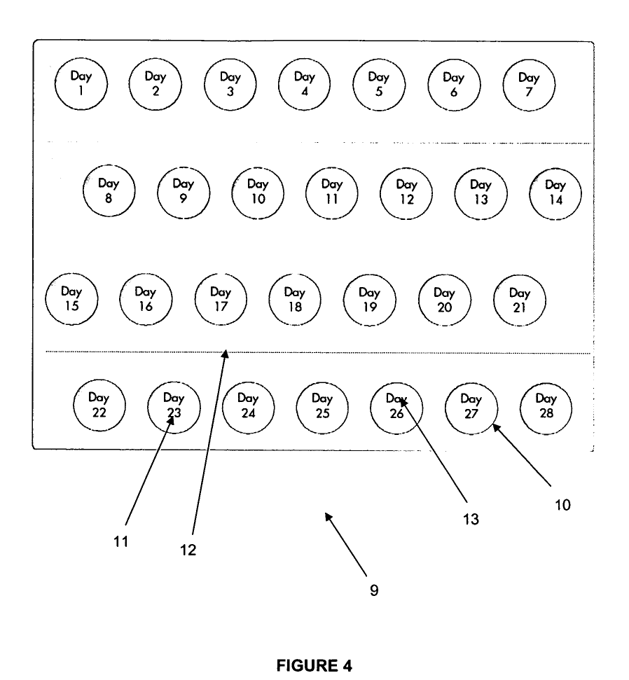

FIG. 3 shows a plan view of a second embodiment of the present invention. The blister pack generally indicated by arrow 4 contains fourteen wells 5 corresponding to a twice-daily dose 6 for the administration of hormone containing cream. A first row 7 is used for the morning application and a second row 8 is used for an evening application.

FIG. 4 shows a further embodiment of the present invention. The blister pack generally indicated by arrow 9 includes twenty eight wells 10 corresponding to a once-per-day dose 11 for the administration of hormone-containing cream. Perforations 12 are included between seven well groupings on the blister pack 9 to allow the user to detach week long sections.

To aid the user to identify which dose to administer, the blister pack 1,4,9 preferably includes indicia to give an indication as to what dose is required to be taken and when.

In one embodiment, a word indicia is used such as that shown in FIGS. 1 to 4 indicated by arrow 13. Words can include the time, for example ‘AM’ or ‘PM’, the day, the week number, the month or the quarter or any other identifier appropriate to the medication to be administered.

In a further embodiment, the blister pack 1,4,9 as a whole or in part, such as a well or wells, are colored (not shown). Preferably the color coding is part of a logical progression such as a rainbow color spectrum or traffic light range of colors to show the order of use/consumption.

In yet a further embodiment, the cream itself provides the color indicator. In this embodiment, the well 2,5,10 is made of a clear material through which the color of the dose of cream 3,6,11 inside can be determined. The cream is colored to reflect a desired indicia, for example the day of the week, the time of the day or the dose concentration. The range of color may be as described above or a progression of light to dark variants of one color. It should be appreciated by those skilled in the art that a wide variety of other color indicia may also be used to reflect the dosage regime.

In preferred embodiments the wells 2,5,10 have a seal (not shown) that is independently accessible, removable or breakable. The key method envisaged of removing the dose 3,6,11 from the blister pack 1,4,9 is to rupture the seal by inverting the well 2,5,10 by hand and thus exposing the dose 3,6,11 on the underside of the well 2,5,10. The dose 3,6,11 can then be removed and applied to the skin.

It should be appreciated by those skilled in the art that the apparatus described, whilst described with respect to cream carrier solutions may also be used in conjunction with tablets and other known carriers. For example, the apparatus of the present invention may be used in conjunction with a tablet whereby a first section of the apparatus contains wells 2,5,10 incorporating a cream dose 3,6,11 of the present invention and a second section incorporates a tablet dose for an associated treatment via, for example, an oral administration method.

The inventors envisage a number of advantages over the prior art for the invention. These include:

Precise doses that are quickly and easily administered by the end user;

Users can instantly tell how many applications/doses have been used and how many are left;

Different ratios/combinations/strengths of active ingredients can be delivered in each dose;

The present invention helps in removing vagaries associated with dosing of creams;

The present invention avoids contamination of the creams (for example creams presented in jars are prone to contamination by fingers);

Combination oral and topical actives can be dispensed in one easy to follow regime;

The present invention is less bulky than jars or tubes of cream and thus can easily be carried discretely.

Aspects of the present invention have been described by way of example only and it should be appreciated that modifications and additions may be made thereto without departing from the scope thereof as defined in the appended claims.

What is claimed is:

1. A disposable dispensing apparatus for storage and dispensing of a topically applied medicament to an animal by a dosing regimen to facilitate administration of uneven doses of a biologically active substance, said apparatus comprising;
a blister pack having a plurality of predetermined medicament-containing wells;
characterised in that the plurality of wells are grouped to contain medicaments, the wells being arranged and grouped for use of the medicament in each well; and,
further characterised in that medicament is incorporated within a solution suitable for topical administration; and,
wherein the medicament contains at least one biologically-active substance.

2. The apparatus as claimed in claim 1 wherein the solution is selected from the group consisting of: cream, gel, suspension, semi-solid solution.

3. The apparatus as claimed in claim 1 wherein the apparatus comprises a group of wells arranged in a plurality of rows of cream containing wells.

4. The apparatus as claimed in claim 1 wherein the wells are grouped for sequential use.

5. The apparatus as claimed in claim 1 wherein the blister pack comprises indicia on the blister packet to indicate when the dose is to be taken.

6. The apparatus as claimed in claim 5 wherein the indicia are different colors.

7. The apparatus as claimed in claims 1 wherein at least the top of at least one well is a transparent material and the solution colors are used as indicia.

8. The apparatus as claimed in claim 7 wherein each aqueous solution color is used to indicate elements selected from the group consisting of: the biologically-active substance to be taken, the biologically-active substance concentration and/or volume, the stage of progress through a specified treatment, time of day, day, week, month, quarter, and combinations thereof.

9. The apparatus as claimed in claim 8 wherein the colors used form a recognisable sequence of colors.

10. The apparatus as claimed in claim 1 wherein the dose to be taken is varied depending on the biologically-active substance to be taken, time of day, day, week, month, quarter.

11. The apparatus as claimed in claim 1 wherein the apparatus has time indicia comprising markings selected from the group consisting of: AM, PM, morning, daytime, night-time, and combinations thereof.

12. The apparatus as claimed in claim 1 wherein the apparatus has day indicia comprising markings selected from the group consisting of: Monday, Tuesday, Wednesday, Thursday, Friday, Saturday, Sunday, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, one, two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one and combinations thereof.

13. The apparatus as claimed in claim 1 wherein the apparatus has week indicia comprising markings selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and combinations thereof.

14. The apparatus as claimed in claim 1 wherein the apparatus has month indicia comprising markings selected from the group consisting of: J, F, M, A, M, J, J, A, S, O, N, D, Jan, Feb, Mar, Apr, May, Jun, Jul, Aug, Sep, Oct, Nov Dec, January, February, March, April, May, June, July, August, September, October, November, December, and combinations thereof.

15. The apparatus as claimed in claim 1 wherein the apparatus has quarter indicia comprising markings selected from the group consisting of: 1,2,3,4, JFM, AMJ, JAS, OND, and combinations thereof.

16. The apparatus as claimed in claim 1 wherein each well has a seal that is independently accessible, removable or breakable.

17. The apparatus as claimed in claim 1 wherein groups of wells include surrounding perforations.

18. The apparatus as claimed in claim 1 wherein the blister pack also includes non-aqueous solution medicaments.

19. The apparatus as claimed in claim 1 wherein the apparatus further includes a container for receiving and storing a blister pack or packs.

20. The apparatus as claimed in claim 1 wherein the container is divided into a plurality of discrete compartments and wherein at least one compartment is for receiving and storing a blister pack and at least one compartment is for receiving and storing further blister packs.

21. The apparatus as claimed in claim 1 wherein the blister pack is configured in a rolled formation.

22. The apparatus as claimed in claim 1 wherein the animal is a human.

23. The apparatus as claimed in claim 1 wherein the medicament within the wells contains an uneven dose of one of four or more different varieties of concentration, type or volume of biologically-active compound.

24. The apparatus as claimed in claim 1 wherein the dose taken is one well per day.

25. The apparatus as claimed in claim 1 wherein the blister pack contains doses of medicament in each well tailored to the dosage required by the patient.

26. The apparatus as claimed in claim 1 wherein the total number of doses included on a blister packet is for a twenty-eight day time period.

27. The apparatus as claimed in claim 1 wherein the total number of doses included on a blister packet is for a thirty day time period.

28. The apparatus as claimed in claim 27 wherein the biologically-active substances are hormones and wherein the quantity of medicament available in each successive well is sufficient for the daily needs of a woman's natural monthly cycle.

29. A disposable dispensing apparatus for storage and dispensing of a topically applied medicament to an animal by a dosing regimen to facilitate administration of uneven doses of a biologically active substance, said apparatus comprising;
a blister pack having a plurality of predetermined medicament-containing wells;
characterised in that the plurality of wells are grouped to contain medicaments, the wells being arranged and grouped for use of the medicament in each well;
the medicament is incorporated within a solution suitable for topical administration and the medicament contains at least one biologically-active substance; and
further characterised in that the blister pack comprises indicia on the blister packet, the idicia being of different colors, to indicate when the dose is to be taken.

30. A disposable dispensing apparatus for storage and dispensing of a topically applied medicament to an animal by a dosing regimen to facilitate administration of uneven doses of a biologically active substance, said apparatus comprising;
a blister pack having a plurality of predetermined medicament-containing wells;
characterised in that the plurality of wells are grouped to contain medicaments, the wells being arranged and grouped for use of the medicament in each well;
the medicament is incorporated within a solution suitable for topical administration and the medicament contains at least one biologically-active substance; and
further characterised in that at least part of the top of at least one well is a transparent material and the solution colors are used as indicia.
